(19) United States
(12) Patent Application Publication
(10) Pub. No.: US 2008/0147037 A1
(43) Pub. Date: Jun. 19, 2008

(54) System for withdrawing and dispensing liquid medication from a medication container

(76) Inventor: Kevin L. Foster, Pasadena, CA (US)

Correspondence Address: Frederick Gotha, Esq., Suite 823, 80 South Lake Avenue, Pasadena, CA, 91101, US

(21) Appl. No.: 11/639,641

(22) Filed: Dec. 15, 2006

(51) Int. Cl. A61B 19/00

(52) U.S. Cl. 604/415

(57) ABSTRACT

The invention relates to an improved system for withdrawing and dispensing a measured dose of liquid medication from a medication container and includes a medication container of the type having a bottle neck with a void therethrough communicating with the reservoir of the container and a suction syringe having a needle portion through which the medication is suctioned by the syringe. An improved neck insert has a first passageway passing therethrough and a second passageway passing therethrough where the second passageway communicates with the atmosphere and the container reservoir; the first passageway is so dimensioned and proportioned to sealingly receive the needle portion of the syringe. The improved neck insert is resiliently compressible radially and peripherally seals the bottle neck void upon sufficient insertion into the void. A retrieving tube has a distal portion and a proximate portion and a lumen extending therethrough. The proximate portion of the retrieving tube is carried by the neck insert and the distal portion of the retrieving tube communicates with liquid medication thereby providing a flow path for the liquid medication into the suction syringe.

### FIELD OF THE INVENTION

This invention relates to an improved system for withdrawing a desired dosage of liquid medication from the reservoir of a medication container.

### BACKGROUND OF THE INVENTION

The use of a suction type syringe to withdraw a desired dosage of liquid medication or serum from a bottle is known in the prior art. Typically, the neck of the bottle is circumferentially sealed by a press-fit plug and the syringe needle portion is pressed into a narrow opening extending through the plug. The plug is made of a resilient material, such as rubber, that has the physical property of elasticity under radial compression; consequently the needle portion of the syringe is radially compressed after insertion through the narrow opening which ideally permits the bottle to be inverted with limited leakage of fluid past the inserted needle portion. Inversion of the bottle is necessary to withdraw the medication into the syringe. Before inversion, however, the plunger of the syringe must be pushed completely downward to force air within the syringe into the bottle. The bottle is then inverted and the medication is withdrawn by pulling the plunger until the desired dosage of medication has been withdrawn into the reservoir chamber of the syringe. Because of the residual air which is present in the syringe reservoir, an accurate dosage is generally unattainable without withdrawing an excess of medication while the bottle remains inverted. The excess is forced back into the bottle by the plunger which also removes the air from the dosage resulting in a more accurate dosage measurement. The bottle container is then turned upright and the syringe removed. Dispension of the medication is then achieved by depressing the plunger.

It is therefore desirable to have a system for withdrawing and administering a desired dosage of medication without inverting the medication container; to have a system that eliminates spillage or leakage of the medication from the container or syringe, and for providing an accurate dosage upon withdrawal of the syringe plunger which thereafter can be administered by depressing the plunger.

### SUMMARY OF THE INVENTION

The present invention relates to an improved system for withdrawing a desired dosage of medication form a bottle container without inverting the bottle thereby averting or minimizing the spillage and leakage that accompanies inversion.

The improved system for withdrawing a desired dosage of medication by a suction syringe consists of a medication container having a bottle neck which when uncapped may be circumferentially sealed by a removably mounted insert or plug that is made of an elastic material having the physical property of being resiliently compressible in a radial direction. The removably mounted insert has an axis of elongation and is so dimensioned and proportioned to circumferentially seal the bottle neck upon insertion, at least in part, therein. A first passageway extends axially through the insert where the passageway is so dimensioned and proportioned to receive the needle portion of a suction syringe such that the needle portion is at least in part radially compressed to permit the needle portion to be in sealed relationship with the insert. A second passageway extends axially through the insert and is radially removed from the first passageway where the second passageway provides an open conduit to the atmosphere while also communicating with the reservoir region of the bottle container. The insert carries a retrieving tube having a lumen extending therethrough where the lumen is in fluid communication with the first passageway. The retrieving tube extends from the insert into the reservoir and is sufficiently dimensioned to permit a portion of the tube to be disposed beneath the surface of the medication during the withdrawal of the dosage from the bottle container.

In one embodiment of this invention, the suction syringe has a plunger for withdrawing the medication into the reservoir chamber of the syringe. In another embodiment, the suction syringe utilizes a bellows to withdraw the medication into the syringe reservoir.

### BRIEF DESCRIPTION OF THE DRAWINGS

These and other features and advantages will become appreciated as the same become better understood with reference to the following specification, claims, and drawings wherein:

FIG. 1 is an illustration of a typical medication container with the bottle cap removed.

FIG. 2 is a cross-sectional view illustrating one embodiment of the system of this invention utilizing a bellows type suction syringe.

FIG. 3 is a cross-sectioned view illustrating another embodiment of the system of this invention utilizing a plunger type suction syringe.

FIG. 4 is a perspective view of the removable insert, illustrating the upper surface of the insert with the medication retrieval tube, extending axially from the lower surface of the insert.

FIG. 5 is a cross-sectional view of the removable insert and medication retrieval tube illustrated in FIG. 4.

### DETAILED DESCRIPTION

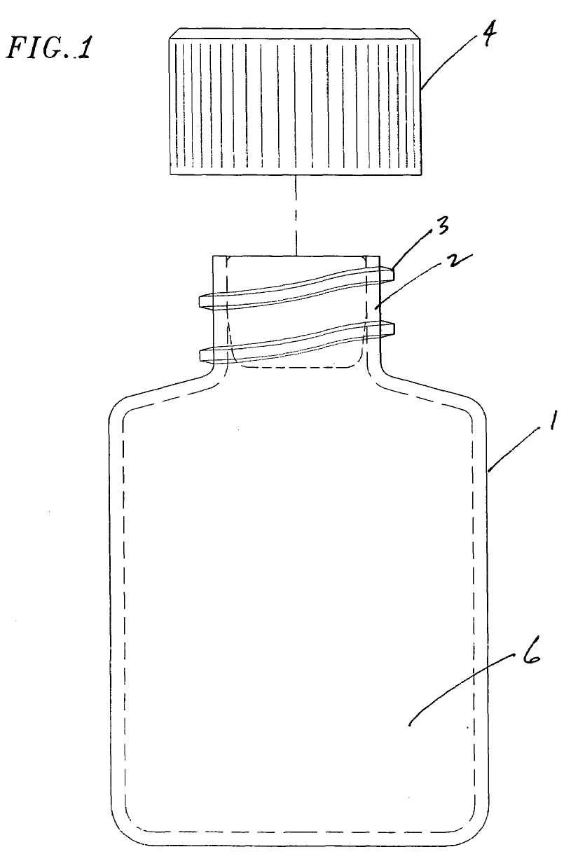

FIG. 1 illustrates a typical medication container which may contain a liquid medicine of which prescribed dosages for children and adults are usually recommended on the label of the container or set forth by the pharmacist when the medication is prescribed by a physician. Accurate measurement of the prescribed or recommended dosage is ordinarily achieved by utilizing a suction syringe which typically is of a bellows or plunger type. When very young children are to receive the medication, it is desirable that the measured dosage be substantially accurate and easily administered. The system of this invention provides an accurate syringe measurement which is easily obtained without dripping and leakage of the medication.

FIG. 1 illustrates a typical medication container 1 which has a bottle-neck 2 where the bottle-neck is threaded 3 for threadably receiving and removing container cap 4. The medication is carried by the medication container in the reservoir region 6.

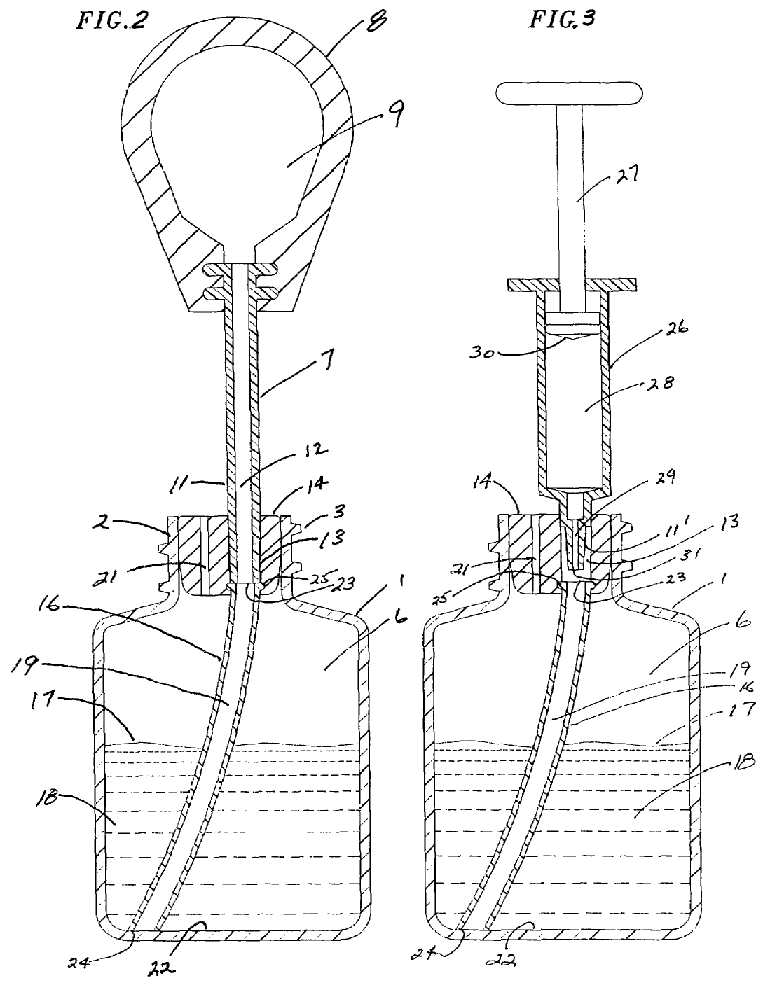

By referring to FIG. 2, one embodiment of the system of this invention can be seen which utilizes a bellows-type suction syringe 7. Bellows-type suction syringes are well known in the prior art and contain a bellows member 8 having an air chamber 9 therein. A needle portion 11 having a conduit 12 extends through the needle portion and communicates with air chamber 9. Needle portion 11 is insertable into first passageway 13 which extends axially through the neck insert 14 where neck insert 14 is made of an elastic material having the physical property of being resiliently compressible in a radial direction. To circumferentially seal bottle neck 2 of medication container 1, neck insert 14 is forceably inserted into the void of bottle neck 2 and is so dimensioned and proportioned to compressibly bear against the inner cylindrical surface wall of bottle neck 2. As can further be seen by reference to FIG. 2, a retrieving tube 16 is carried by neck insert 14 and extends from the lower surface of neck 14 into reservoir region 6 and is so dimensioned and proportioned such that second end 24 of retrieving tube 16 extends beneath and remains below the surface 17 of the medication 18 contained in reservoir region 6. Lumen 19 extends axially completely through retrieving tube 16, and communicates with both first passageway 12 and medication liquid 18, thereby providing a fluid path for medication 18 into the first passageway. A second passageway 21 extends axially through neck insert 14 and communicates with both reservoir region 6 and the atmosphere thereby maintaining the liquid surface 17 of the medication under atmospheric pressure at all times.

To withdraw a desired dosage of medication utilizing the system embodiment illustrated in FIG. 2, container cap 4 is first removed from medication container 1 and thereafter neck insert 14 is forceably inserted into the void of bottle neck 2 circumferentially sealing the inner cylindrical wall of bottle neck 2. Retrieving tube 16 has a first end 23 and a second end 24 where second end 24 after insertion extends below the surface of the medication and preferably is positioned adjacent to the bottom 22 of reservoir region 6. The needle portion 11 of bellow syringe 7 is then forcefully pressed into first passageway 13 and as a result thereof is circumferentially sealed by the radial compression exerted by neck insert 14. Needle portion conduit 12 which extends through needle portion 11 is in fluid communication with lumen 19 after needle portion 11 is at least in part in radial compressive engagement with neck insert 14 thereby providing a flow path for the liquid medication from the reservoir. Second passageway 21 which is vented to the atmosphere allows air which has been forced into the reservoir by depressing bellows member 8 to be vented to the atmosphere while the pressure acting on the surface 17 remains at atmospheric. The pressure within bellows air chamber 9 is equal to the atmosphere plus the height of the medication within the container. By allowing the bellows member 9 to expand, the increased volume of bellows member 9 results in decreasing the pressure therein and consequently the liquid medication will flow into the needle portion. When an excess of the desired dosage is drawn into the needle portion, the bellows member may again be depressed forcing the excess back to the reservoir until the desired dosage has been determined. The needle portion is then withdrawn and the bellows permitted to expand reducing the pressure within the bellows air chamber to less than atmosphere thereby permitting atmospheric pressure to act upon the column of medication withdrawn into the syringe and prevent it from escaping until the bellows is again depressed.

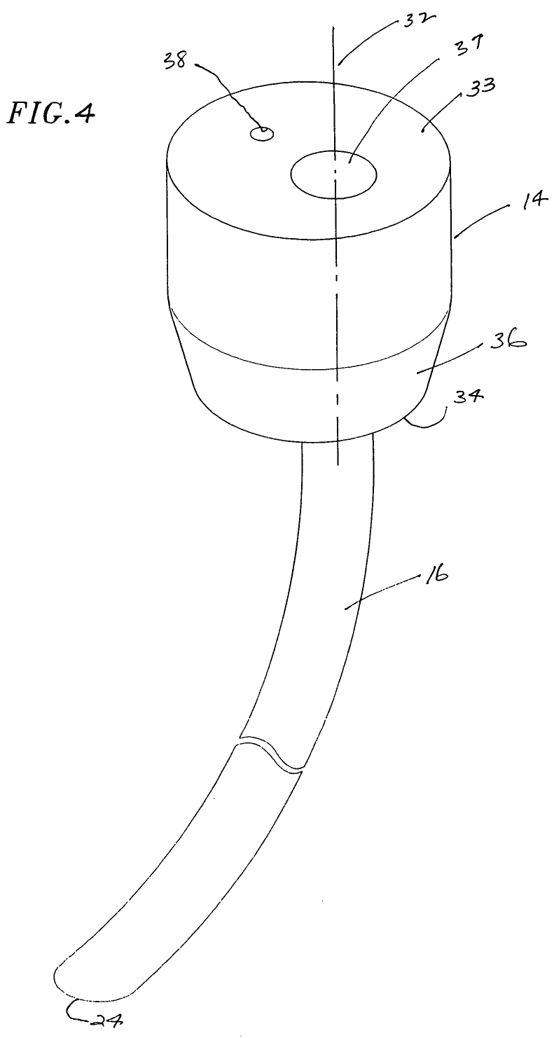

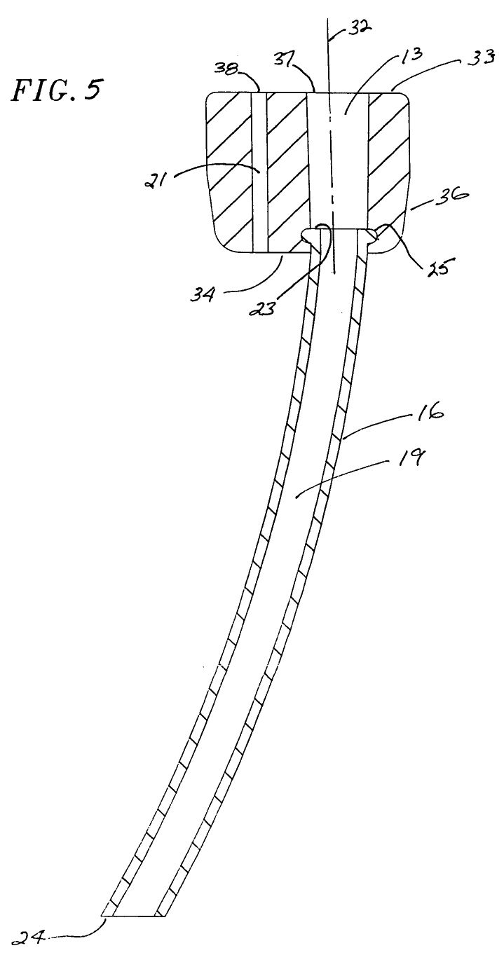

Another embodiment of the system of this invention is shown in FIG. 3. As can be seen in FIG. 3, the embodiment illustrated therein differs from the embodiment described above only by the use of a plunger syringe 26 rather than the bellows syringe 7 depicted in FIG. 2. Plunger syringe 26 has a plunger member 27 that carries a piston 37 sealingly and slidably carried within cylinder chamber 28. Conduit 29 is in fluid communication with and communicates with cylinder chamber 28 and provides a fluid passageway through the needle portion 11 of the syringe. Opening 31 at the distal end of needle portion 11 communicates with first passageway 13 of neck insert 14. As in the previous system embodiment, neck insert 14 has a second passageway 21 which is vented to the atmosphere and extends axially through neck insert 14 and communicates with the reservoir region 6 of the medication container. FIG. 4 and FIG. 5 illustrate neck insert 14 with retrieving tube 16 carried in fluid relationship with the insert.

Referring to FIG. 4, neck insert 14 can be seen in perspective with retrieving tube 16 extending axially therefrom. As can further be seen in FIG. 5, neck insert 14 has an axis of elongation 32, an upper surface 33 and a lower surface 34 which preferably has a surface area less then the surface area of upper surface 33. Neck insert 14 preferably has a cylindrical shape and contains a tapered region adjacent lower surface 34 to assure a circumferential seal when neck insert 14 is forcefully inserted into bottle neck 2 of medication container 1. Upper surface 33 has openings 37 and 38 where opening 37 communicates with first passageway 13 and opening 38 communicates with second passageway 21. By again referring to FIG. 5 (which is a cross-sectional view of FIG. 4), the venting second passageway 21 can clearly be seen extending axially through neck insert 14; the liquid passageway communication between the lumen 19 and first passageway 13 is also clearly illustrated. Although first end 23 of retrieving tube 16 is shown having lip portion 25 removably coupled to neck insert 14, retrieving tube 16 could also be integrally carried by neck insert 14.

The embodiment of the system of this invention utilizing a plunger type suction syringe permits a measured dosage of medication to be withdrawn in a like manner as that described for the bellows syringe. The needle portion 11 is forcefully inserted into first passageway 13 of neck insert 14 where the needle portion is sealed by the radial compression exerted by the resilient radial expansion of first passageway 13. By depressing plunger 27, the air within cylinder chamber 28 is exhausted to the atmosphere through second passageway 21. Upon withdrawal of the plunger, the pressure within cylinder 28 is less than atmosphere and the medication will be drawn into the chamber without inverting the container. Upon withdrawal of the needle portion from the first passageway, the difference between atmospheric pressure and the pressure within cylinder chamber 28 will exert a force at opening 31 of conduit 29 that will prevent leakage of the dosage from the syringe.

While I have shown and described embodiments of an improved system for withdrawing a measured dosage of medication from a container bottle, it is understood that the invention may be subject to many modifications without departing from the scope and spirit of the claims as recited herein.

What is claimed is:

1. An improved system for withdrawing and dispensing a measured dosage of liquid medication from the reservoir region of a medication container, said medication container having a bottle neck extending therefrom and where said bottle neck has a void therethrough communicating with said reservoir region comprising:
(a) a suction syringe for withdrawing and dispensing said liquid medication;
(b) a needle portion carried by said suction syringe;
(c) a neck insert made of a resilient material having the physical property of being resiliently compressible radially, said neck insert having an axis of elongation, a first passageway extending axially therethrough and a second passageway extending axially therethrough, where said first passageway is so dimensioned and proportioned to permit said needle portion to sealingly couple therewith upon sufficient axial extension of said needle portion within said first passageway, and where said second passageway has an opening communicating with the atmosphere, and where said neck insert is so dimensioned and proportioned to peripherally seal said bottle neck upon sufficient axial displacement within said void;
(d) a retrieving tube, having a proximate portion and a distal portion, an axis of elongation and an axially extending lumen therethrough where said proximate portion is carried by said neck insert and said lumen communicates with said first passageway and where said distal portion is disposed within said liquid medication so as to provide a fluid path for said liquid medication from said medication container into said suction syringe.

2. The system recited in claim 1 where said suction syringe is a bellows-type syringe.

3. The system recited in claim 1 where said suction syringe is a plunger-type syringe.

4. The system recited in claim 2 where said bottle neck is substantially cylindrical.

5. The system recited in claim 3 where said bottle neck is substantially cylindrical.

6. The system recited in claim 2 where said retrieving tube is removably carried by said neck insert.

7. The system recited in claim 3 where said retrieving tube is removably carried by said neck insert.

8. The combination for withdrawing and dispensing a measured dosage of liquid medication comprising:
(a) medication container having a reservoir region, said medication container of the type having a bottle neck extending therefrom where said bottle neck has a void extending therethrough and communicating with said reservoir region;
(b) a suction syringe for withdrawing and dispensing said liquid medication;
(c) a needle portion carried by said suction syringe;
(d) a neck insert made of a resilient material having the physical property of being resiliently compressible radially, said neck insert having an axis of elongation, a first passageway extending axially therethrough and a second passageway extending axially therethrough, where said first passageway is so dimensioned and proportioned to permit said needle portion to sealingly couple therewith upon sufficient axial extension of said needle portion within said first passageway, and where said second passageway has an opening communicating with the atmosphere, and where said neck insert is so dimensioned and proportioned to peripherally seal said bottle neck upon sufficient axial displacement within said void;
(e) a retrieving tube having a proximate portion and a distal portion, an axis of elongation, and an axially extending lumen therethrough where said proximate portion is carried by said neck insert and said lumen communicates with said first passageway and where said distal portion is disposed within said liquid medication so as to provide a fluid path for said liquid medication from said medication container into said suction syringe.

9. The combination recited in claim 8 where said suction syringe is a bellows-type syringe.

10. The combination recited in claim 8 where said suction syringe is a plunger-type syringe.

11. The combination recited in claim 9 where said bottle neck is substantially cylindrical and said void is substantially cylindrical.

12. The combination recited in claim 10 where said bottle neck is substantially cylindrical and said void is substantially cylindrical.

13. The combination recited in claim 9 where said retrieving tube is removably carried by said neck insert.

14. The combination recited in claim 10 where said retrieving tube is removably carried by said neck insert.

15. An improved insert device of the type for insertion into the bottle neck of a liquid medication container said bottle neck having a void therethrough where the liquid medication is withdrawn from the medication container reservoir through the needle portion of a suction syringe, said needle portion in fluid communication with said reservoir, the improvement comprising:
(a) a neck insert made of a resilient material having the physical property of being resiliently compressible radially, said neck insert having an axis of elongation, a first passageway extending axially therethrough and a second passageway extending axially therethrough, where said first passageway is so dimensioned and proportioned to permit said needle portion to sealingly couple therewith upon sufficient axial extension of said needle portion within said first passageway, and where said second passageway has an opening communicating with the atmosphere, and where said neck insert is so dimensioned and proportioned to peripherally seal said bottle neck upon sufficient axial displacement within said void;
(b) a retrieving tube, having a proximate portion and a distal portion, an axis of elongation and an axially extending lumen therethrough where said proximate portion is carried by said neck insert and said lumen communicates with said first passageway and where said distal portion is disposed within said liquid medication so as to provide a fluid path for said liquid medication from said medication container into said suction syringe.

16. The improved insert device recited in claim 15 where said neck insert is cylindrically shaped and where said distal portion is conically tapered axially.
